(19) United States
(12) Patent Application Publication
(10) Pub. No.: US 2014/0044652 A1
(43) Pub. Date: Feb. 13, 2014

(54) Methods and Related Systems for Selecting or Customizing a Topical Treatment Regimen for a Mammalian Keratinized Surface

(71) Applicant: 3000BC LLC, (US)

(72) Inventors: KORIN KORMAN, FORT WASHINGTON, PA (US); RODMAN HECKMAN, CHESTER SPRINGS, PA (US)

(73) Assignee: 3000BC LLC, Philadelphia, PA (US)

(21) Appl. No.: 13/887,951

(22) Filed: May 6, 2013

Related U.S. Application Data
(60) Provisional application No. 61/642,653, filed on May 4, 2012.

(51) Int. Cl. A61K 38/18; A61Q 19/10; A61K 8/99; A61Q 19/00; A61Q 17/04; A61K 31/375

(52) U.S. Cl. CPC A61K 38/1841; A61Q 17/04; A61K 31/375; A61K 8/99; A61Q 19/007; A61Q 19/10; A61Q 19/00
USPC 424/59; 514/8.9; 514/474; 424/195.17

(57) ABSTRACT

The invention described herein includes a method of selecting a treatment regimen for a surface of a mammalian keratinized tissue that includes illuminating the surface with a light source emitting long 5 wave UV light, thereby identifying at least one impaired region on the surface; categorizing the impaired region on the basis of its approximate UV perceptible color when exposed to the light source into at least one of a first category, a second category and a third category, wherein the approximate color substantially corresponds to a deficiency of the surface; and applying to the keratinized surface at least one treatment composition, wherein the 10 at least one treatment composition is formulated to treat, ameliorate or improve the deficiency in the surface.

### CROSS REFERENCE TO RELATED APPLICATIONS

This application claims the benefit under 35 U.S.C. §119(e) of U.S. Provisional Application No. 61/642,653, filed May 4, 2012, entitled Methods and Related Systems for Selecting or Customizing a Topical Treatment Regimen for a Mammalian Keratinized Surface, the contents of which are incorporated herein by reference.

### BACKGROUND OF THE INVENTION

Skin is the largest organ of the human body; it comprises about one-sixth of the total body weight. It protects against ultraviolet rays; from mechanical, chemical and thermal injuries; from bacterial invasions and from dehydration and water penetration. Facial skin in particular is exposed to such elements on a daily basis, as it is rarely covered by clothing or other UV or environmental barriers. Accordingly facial skin may be come sun damaged, dehydrated or suffer other detriments as a result of ordinarily exposure.

To repair, prevent or ameliorate this damage, individuals spend significant time and resources attempting to indentifying suitable treatment regimens and products to apply to one's skin. Because every individual has a different skin type and suffers from different types of damages or disorders, identifying a suitable treatment regimen may be time consuming, expensive and/or difficult. Thus, there remains a need in the art for an easy to use method of indentifying a skin care regimen for an individual.

### BRIEF DESCRIPTION OF THE SEVERAL VIEWS OF THE FIGURES

The foregoing summary, as well as the following detailed description of preferred embodiments of the invention, will be better understood when read in conjunction with the appended drawings. However, it should be understood that the invention is not limited to the precise arrangements and instrumentalities shown. In the drawings:

FIG. 1 is a photograph of an individual exposed to the UV light source showing impaired regions of a UV perceptible region that appear yellow;

FIG. 2 is a photograph of an individual exposed to the UV light source showing impaired regions of a UV perceptible region that appear brown;

FIG. 3 is a photograph of an individual exposed to the UV light source showing impaired regions of a UV perceptible region that appear purple; and

FIGS. 4, 5, and 6 are photographs illustrating a process for determining a predominant color.

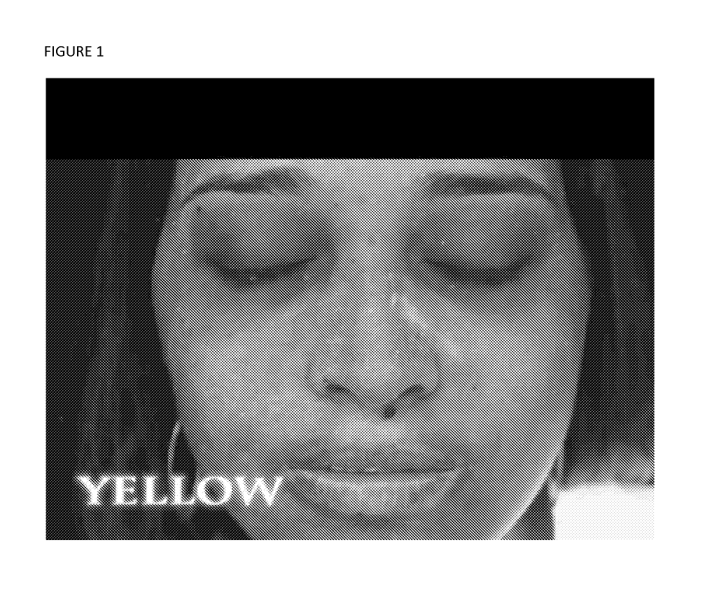

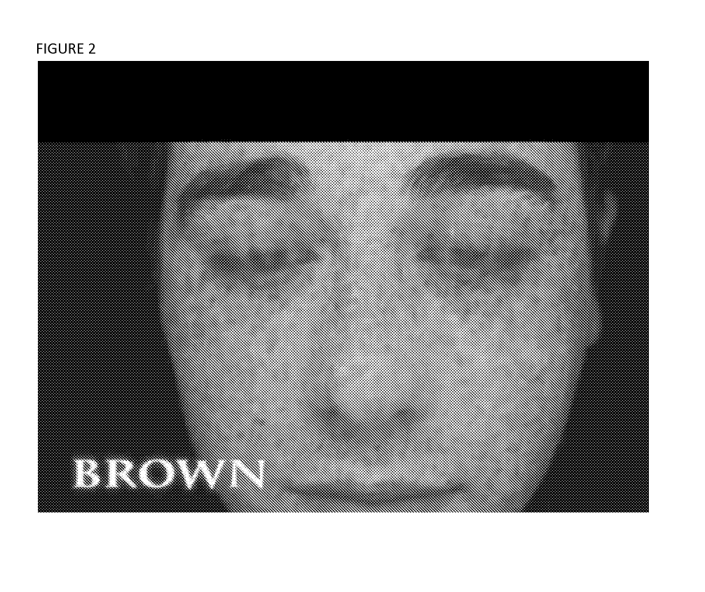

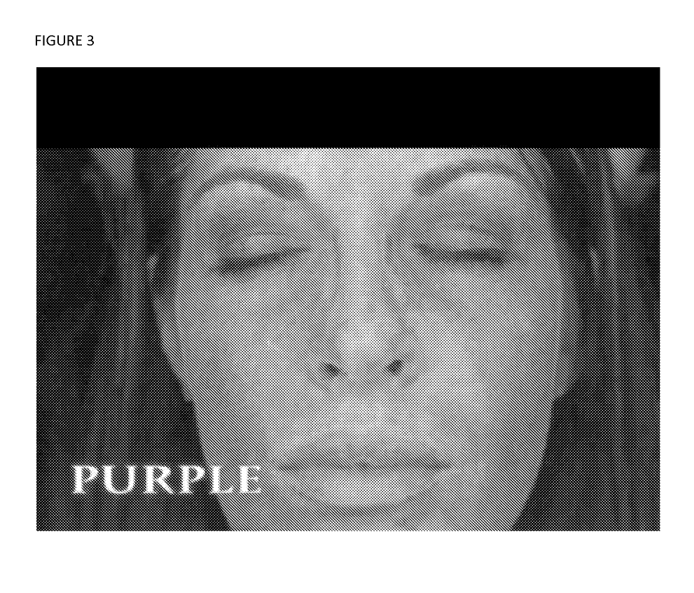

### BRIEF DESCRIPTION OF THE INVENTION

The invention described herein includes a method of selecting a treatment regimen for a surface of a mammalian keratinized tissue that includes illuminating the surface with a light source emitting long wave UV light, thereby identifying at least one impaired region on the surface; categorizing the impaired region on the basis of its approximate UV perceptible color when exposed to the light source into at least one of a first category, a second category and a third category, wherein the approximate color substantially corresponds to a deficiency of the surface; and applying to the keratinized surface at least one treatment composition, wherein the at least one treatment composition is formulated to treat, ameliorate or improve the deficiency in the surface.

The invention may further encompass a customized system for the treatment of the skin of an individual, the treatment system comprising at least two compositions, wherein the compositions are selected by illuminating the surface with a light source emitting long wave UV light, thereby identifying at least one impaired region on the surface; categorizing the impaired region on the basis of its approximate UV perceptible color when exposed to the light source into at least one of a first category, a second category and a third category, wherein the approximate color substantially corresponds to a deficiency of the surface; and providing at least one treatment composition, wherein the at least one treatment composition is formulated to treat, ameliorate or improve the deficiency in the surface.

### DETAILED DESCRIPTION OF THE INVENTION

The invention described herein includes methods of selecting, optimizing, customizing, and/or developing a treatment regimen for the surface of a mammalian keratinized tissue. Also included are the treatment systems customized for an individual developed through the use of the methods.

The invention permits the development of a skin care regimen that is specifically tailored to address the particular needs or deficiencies of a given keratinized surface(s). Thus, an individual's minor (that is, not acutely medically concerning) skin care needs may be addressed in a targeted manner. Such methods arc advantageous as they are less time and cost intensive than seeking formal dermatological treatments for minor skin care deficiencies that can be address via botanical and/or other over-the-counter treatments. The methods are also advantageous because they are more accurate than other common methods. In the practice of the methods, the deficiencies are indentified by illuminating the surface of the keratinized tissue with a light source emitting UV light. The keratinized tissue may include, for example, the hair, the nails and/or the skin. If skin is the tissue selected, the skin may be on any part of the body and may include, without limitation, skin of the hand, neck face, and/or upper chest (décolleté).

In some embodiments of the invention, the UV-emitting light sources emits long wave UV light, for example, without limitation, UV light having a wavelength of about 400 to about 300 nanometers or UV light having a wavelength of about 400 to about 315 nanometers. While a simple, single bulb light sources may be used, other more complex light source devices are also contemplated within the scope of the invention, including, without limitation, the SkinScope.

In the practice of the methods, the keratinized surface, for example, the skin of the face, is exposed to UV light. Preferably, the UV light exposure is carried out under reduced or the absence of visible light, to allow for more optimal viewing of any impaired regions on the skin surface. In one embodiment, the light source is placed in a box or tent or other structure that serves to substantially block visible light, and the portion of keratinized tissue to be analyzed in placed within the structure before illumination. In some embodiments of the invention, the UV light exposure may be carried out in front of a reflective surface (such as a conventional mirror) and camera or a video camera, so the individual upon whom the method is being practiced may observe in real time and/or keep a record of the images.

Upon illumination with the light source, most keratinized tissue surfaces will appear washed in a light blue or violet hue. Against this pastel field appear substantially discrete regions of another, more saturated color (“UV perceptible color”), each of which represents an impaired region on the surface or within the keratinized tissue. The UV perceptible color of the impaired region may correspond to an impairment or deficiency of the region. For example, in an embodiment, the deficiencies may be sun damaged tissue, prematurely aged tissue, dehydrated tissue, dead cells or horny tissue, and tissues coated with excess oils or sebum.

In an embodiment of the invention, the UV perceptible color of the substantially discrete regions of the UV perceptible color may be brown, purple and yellow, and they may correspond to the deficiencies of prematurely aged tissue, dehydrated tissue, and excess oil on the tissue surface, respectively. Other UV perceptible colors may be perceived, depending on the nature and/or wavelength of the light source or light source device that is used in the process.

Each of the impaired regions is categorized into a first category, a second category, and/or a third category on the basis of the region's UV perceptible color(s) under exposure to the UV-emitting light source. For example, in an embodiment, an individual's facial skin is illuminated, and seven UV perceptible regions are visible. Such regions are categorized on the basis of their UV perceptible color, with like colors being categorized together. Thus, in the example, two of the seven color-saturated regions are white-colored, three are purple-colored and two are brown-colored, and each of the regions is categorized by its color. In alternative embodiments, the category(ies) may be given a tag, name, or identifier, such as a number, a letter, a motto, a symbol, a mascot, or color name.

To customize the skin treatment regimen, topical compositions expressly developed for the deficiency to which the identified category corresponds are applied. In the event that the impaired regions are classified into more that one category, a topical composition expressly developed for the deficiency to which the predominant category corresponds are applied. A category is considered to be “predominant” when the corresponding impaired region(s) substantially cover about 30% to about 40% of the surface area that is being illuminated, even if other impaired regions (falling into other categories) are identified.

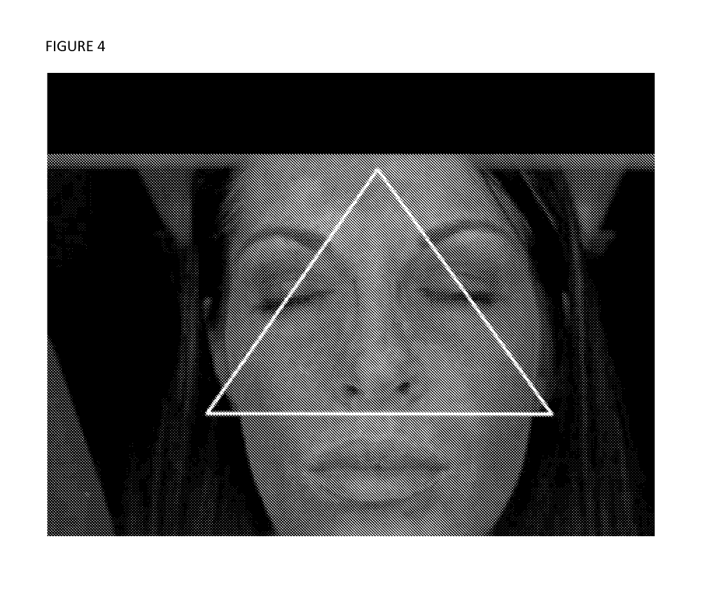

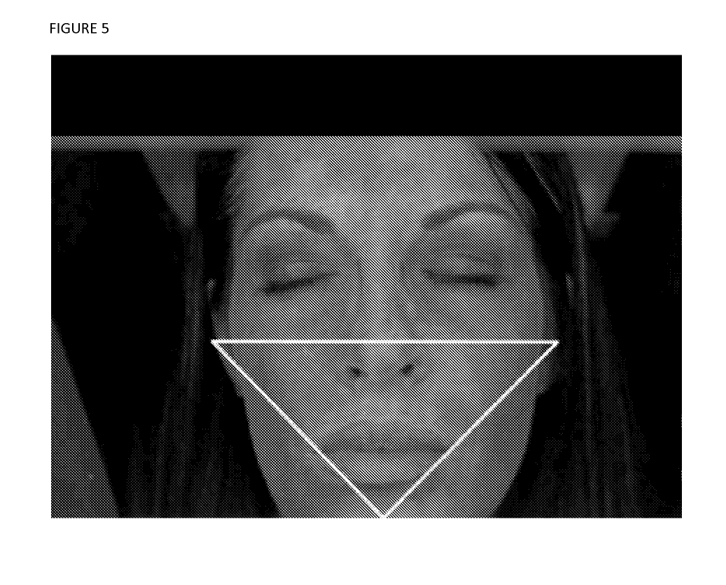

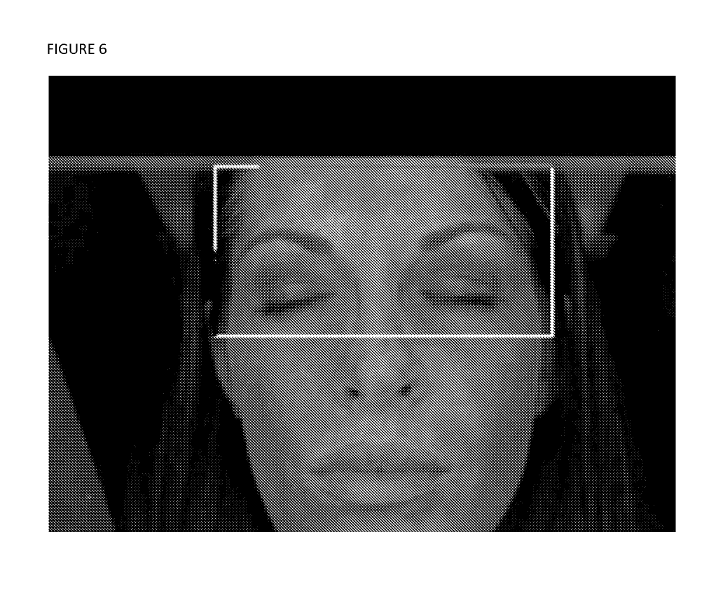

In an exemplary embodiment of the practice of the methods, the illuminated surface may be a facial surface, and the impaired regions are substantially present in a triangular shaped region or an oval or elliptical shaped region, as can be seen in FIG. 4. Alternatively, the illuminated surface may be a facial surface, and the impaired regions are substantially present in a rectangular shaped region on the surface. See, FIG. 6.

Once the category of an impaired region(s) or predominant region(s) is determined, is categorized, one or more topical compositions is applied to this surface. It may generally be preferred that the topical composition(s) is applied to the entire surface. However application may be made to less than the entire surface, for example, without limitation, to about 20%, about 30%, about 40% or up to about 90% of the entire surface (by area).

The topical compositions are to treat, ameliorate or improve the deficiency in the surface, that is, to address the deficiency that corresponds to the identified category or predominant category. For example, if the category or predominant category indentified corresponds to impaired regions of a brown color which corresponds to an indentified deficiency of prematurely aged tissue, the at least one composition applied may be a defense composition. In an embodiment, the defense composition may contain, for example, a therapeutically effective amount of TGF-beta-1 and/or, optionally, vitamin C. Such components may be present in any amount in the compositions so long as the amount is therapeutically effective. As is known in the art, an amount that is “therapeutically effective” may vary depending on a variety of factors, including the ingredient, the bioavailability of the form of the ingredient, and/or the adjuvant or delivery system of the ingredient. Determination of a “therapeutically effective amount” is well within the routine skill of a person of skill in the art. Suitable amounts may include, for example, 10%, 20%, 30%, 40%, 50%, 60%, 70%, 80%, and/or 90% percent by weight of the total composition.

In another embodiment, when the category or predominant category indentified corresponds to impaired regions of a brown color which in turn corresponds to an indentified deficiency of prematurely aged tissue, more than one composition may be applied, for example, one or more of a cleansing composition, a repair composition, a moisturizing composition or a sunscreen composition may be applied. Such compositions are described herein in further detail.

In another embodiment of the invention, the category or predominant category indentified corresponds to impaired regions of a yellow color which in turn corresponds to an indentified deficiency of excess oil. In this circumstance, the at least one composition applied may be a repair composition, comprising a therapeutically effective amount of glycolic acid. It may be desirable that the glycolic acid is present in the composition in an amount of, for example, 10%, 20%, 30%, 40%, 50%, 60%, 70%, 80%, and/or 90% percent by weight of the total composition.

In another embodiment, when the category or predominant category indentified corresponds to impaired regions of a yellow color which in turn corresponds to an indentified deficiency of excess oil, more than one composition may be applied, for example, one or more of a cleansing composition, a defense composition, a moisturizing composition or a sunscreen composition may be applied. Such compositions are described herein in further detail.

In another embodiment of the invention, the category or predominant category indentified corresponds to impaired regions of a purple color which in turn corresponds to an indentified deficiency of dehydrated tissue. In this circumstance, the at least one composition applied may be a moisturizing composition, comprising a therapeutically effective amount of an oil. It may be desirable that the oil is present in the composition in an amount of, for example, 10%, 20%, 30%, 40%, 50%, 60%, 70%, 80%, and/or 90% percent by weight of the total composition.

In another embodiment, when the category or predominant category indentified corresponds to impaired regions of a purple color which in turn corresponds to an indentified deficiency of dehydrated tissue, more than one composition may be applied, for example, one or more of a cleansing composition, a defense composition, a repair composition or a sunscreen composition may be applied. Such compositions are described herein in further detail.

Other ingredients that may be present independently or in mixtures, in any of the compositions described herein include, without limitation, retinols, vitamins, mineral, lipids, colorants, opacifiers, fragrance, essential oil, peptides or amino acids, tetrapeptides, copper, arginine, herbal or botanical extract, neem, UV absorbers, Vitamin C, Vitamin K, Vitamin A, Vitamin E, co-Enzyme Q10, hyaluronic acid, agireline, bee sting venom, attenuated botulitum toxin, emblica, antioxidants, alpha hydroxy acids, such as, for example, glycolic acid, lactic acid, malic acid, citric acid, tartanic acids, beta hydroxy acids, such as salicylic acid, emulsifiers, surfactants, preservatives, water, non-aqueous solvents, dispersants, disodium cocoamphodiacetate, glycol distearate, steareth-4, sodium methylcocoyltaurate, hydroxypropyl guar, hydroxypropyltrimodium chloride, PEG-7 glyceryl cocoate, citric acid, glycerine, triethanolamine, carbomer, phospholipids, retinyl palmitate, retinol, green tea, tocopheryl acetate, ascorbyl palmitate, hydrolyzed silk proteins, steardimonium hydroxypropyl, hydrolyzed wheat protein, superoxide dismutase, green tea extract, methylparaben, disodium EDTA, imidazolidinyl urea, lavender essence, cyclomethicone, glycerine, cocoate, citric acid, benzoyl peroxide, silicone polymer, dimethicone, dimethicone copolyol, phospholipids, cimicifuga racemosa extract, caffeine, camellia sinensis, olive oil, aloe vera leaf juice, sunflower seed oil, rose flower water, glycerin, wheatgerm oil, cocoglucoside, coconut alcohol, radish root ferment filtrate, sucrose cocoate, lauryl glucoside, sclerotium gum, smectite, laponite, calophyllum inophyllum seed oil, ginseng root extract, rosemary leaf extract, lecithin, P-anisic acid, xanthan gum, shea butter, borage oils, rose essential oil, spikenard essential oil, ylang ylang, cumin, cinnamon, exfoliant, phenoxyethanol, C₁₂₋₁₅ alkyl benzoate, cetearyl alcohol, glyceryl stearate, ammonium hydroxide, dimethicone, polyacrylamide, C₁₃₋₁₄ isoparaffin, squalene, mucopolysaccharides, PEG-100 stearate, laureth-7, methylparaben, propylparaben, safflower seed oil, algae extract, oat beta glucan, glycerin, methylsilanol mannuronate, phospholipids, retinyl palmitate, arachidyl alcohol, behenyl alcohol, arachidyl glucoside, dipalmitoyl hydroxyproline, borage oil, shea butter, bergamot essential oil, grapefruit essential oil, lemon essential oil, patchouli essential oil, geranium essential oil, palmarosa essential oil, candeia essential oil and sandalwood essential oil, antimicrobials, capryloyl glycine, undecylenoyl glycine, and Japanese honeysuckle extract.

The compositions used in the methods may be any one or more of a cleansing composition, a defense composition, a repair composition, a moisturizing composition, and/or a sunscreen composition.

The cleansing composition may include any ingredients known or developed in the art that serve to mechanically and/or chemically remove dirt, dead skin oily soils, dirt, sweat, sebum and other residues from the skin surface. Exemplary cleansing ingredients may include exfoliating particles and/or surfactants, which bind to the stratum corneum proteins, resulting in diminished ability of the stratum corneum to bind and hold water, and may include anionic surfactants, amphoteric surfactants, synthetic surfactants, nonionic, cationic surfactants and combinations of the same.

The defense composition may include any ingredients known or developed in the art that serve to enhance, promote skin cells, and may include, for example, vitamin C.

The repair composition may include any ingredients known or developed in the art that serve to promote the repair of the cells of the skin, for example, glycolic acid, retinol and/or green tea extract (antioxidants).

The moisturizing composition may include any ingredients known or developed in the art that serve to replace, replenish to maintain moisture in the skin cells, and may include, for example, lipids, proteins, oils and other fats.

The sunscreen compositions may include those that contain mechanical barriers to UV light (e.g., particulates such as zinc oxide, titanium dioxide, talc) and UV-absorbing compounds, such as organic compounds that may absorb, scatter and/or attenuate UV light. D

Any of the compositions described herein may be formulated in any delivery format known or developed in the art. Without limitation, delivery formats may include liquids, aqueous solution, non-aqueous solutions, emulsions, micelles, creams, lotions, suspensions, pastes, gels, powders, glazes, pomades, pre-impregnated wipes, aerosol or non-aerosol sprays. The compositions may be packaged in and/or applied with any delivery device known or to be developed in the art.

Without limitation, suitable packages or delivery devices may include brushes, pads, atomizers, aerosolizers (with or without propellants), tubs, wands, wipes, moisture activated pre-impregnated wipes, roll-ons, sprayers, and the like.

The invention also includes methods in which the category is assigned a unique identifier, such as, for example, a color, a symbol, a fragrance, a number, an alphanumeric code, a word, a texture, a pattern, or a combination thereof and the compositions to be applied arc assigned the same unique identifier. This allows a user of the method to system to identify with relative ease the proper composition for application. For example, in one embodiment, a first category is assigned the identifier or a “rose flower” image, a second category is assigned the identifier of a “jasmine flower” image and a third category is assigned the identifier of a “tulip flower” image. Accordingly, the at least one composition that is formulated to address the deficiency corresponding to the first category may also be identified by a “rose flower” image, e.g., on its label or container; the at least one composition that is formulated to address the deficiency corresponding to the second category may also be identified by a “jasmine flower” image; and the at least one composition that is formulated to address the deficiency corresponding to the third category may also be identified by a “tulip flower” image.

Also contemplated within the scope of the invention are methods of creating a customized skin care regimen for a mammalian individual. Such methods include illuminating a skin surface of the individual with a light source emitting long wave UV light, thereby identifying at least one impaired region on the surface; categorizing the impaired region on the basis of its approximate UV perceptible color when exposed to the light source into at least one of a first category, a second category and a third category, wherein the approximate color substantially corresponds to a deficiency of the surface; and applying to the keratinized surface at least one treatment composition, wherein the at least one treatment composition is formulated to treat, ameliorate or improve the deficiency in the surface. Details of the method are described above.

The invention also encompasses customized systems for the treatment of the skin of an individual mammal, the treatment system comprising at least two compositions, wherein the compositions are selected by illuminating the surface with a light source emitting long wave UV light, thereby identifying at least one impaired region on the surface; categorizing the impaired region on the basis of its approximate UV perceptible color when exposed to the light source into at least one of a first category, a second category and a third category, wherein the approximate color substantially corresponds to a deficiency of the surface; and providing at least one treatment composition, wherein the at least one treatment composition is formulated to treat, ameliorate or improve the deficiency in the surface. Details of the system are described above.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of selecting a treatment regimen for a surface of a mammalian keratinized tissue comprising:
a. Illuminating the surface with a light source emitting long wave UV light, thereby identifying at least one impaired region on the surface;
b. Categorizing the impaired region on the basis of its approximate UV perceptible color when exposed to the light source into at least one of a first category, a second category and a third category, wherein the approximate color substantially corresponds to a deficiency of the surface; and
c. Applying to the keratinized surface at least one treatment composition, wherein the at least one treatment composition is formulated to treat, ameliorate or improve the deficiency in the surface.

2. The method of claim 1, wherein the keratinized surface is at least one of facial skin, skin of the neck, skin of the décolleté, and skin of the hand.

3. The method of claim 1, further comprising applying to the keratinized surfaceabout one to about ten treatment compositions.

4. The method of claim 1, further comprising applying to the keratinized surface one or more of a cleansing composition, a toning composition, a sunscreen composition, an exfoliating composition, a repair composition, a defense composition, and a moisturizing composition.

5. The method of claim 1, wherein the long wave UV light has a wavelength of about 400 to about 300 nanometers.

6. The method of claim 1, wherein the long wave UV light has a wavelength of about 400 to about 315 nanometers.

7. The method of claim 1, wherein the approximate UV perceptible color corresponds to one of yellow, brown, or purple.

8. The method of claim 1, wherein deficiency in the surface is chosen from prematurely aged tissue, dehydrated tissue, and excess oil.

9. The method of claim 1, wherein the wherein the approximate color is brown, and the deficiency is prematurely aged tissue.

10. The method of claim 9, wherein the at least one treatment composition is a defense composition comprising a therapeutically effective amount of TGF-beta-1.

11. The method of claim 10, wherein the defense composition further comprises a therapeutically effective amount of vitamin C.

12. The method of claim 11, wherein the vitamin C is present in an amount of 15% by weight of the total composition.

13. The method of claim 10, further comprising applying at least one of:
a. A cleansing composition;
b. A repair composition;
c. A moisturizing composition; and
d. A sunscreen composition.

14. The method of claim 9, further comprising applying:
a. A cleansing composition;
b. A repair composition;
j. A moisturizing composition; and
k. A sunscreen composition.

15. The method of claim 1, wherein the wherein the approximate UV perceptible color is yellow and the deficiency is excess oil.

16. The method of claim 15, wherein the at least one treatment composition is a repair composition comprising a therapeutically effective amount of glycolic acid.

17. The method of claim 16, further comprising applying at least one of:
a. A cleansing composition;
b. A defense composition;
c. A moisturizing composition; and
d. A sunscreen composition.

18. The method of claim 15, further comprising applying:
a. A cleansing composition;
b. A defense composition;
c. A repair composition;
d. A moisturizing composition; and
e. A sunscreen composition.

19. The method of claim 1, wherein the wherein the approximate color is purple, and the deficiency is dehydrated tissue.

20. The method of claim 19, wherein the at least one treatment composition is a 20 moisturizing composition.

21. The method of claim 19, wherein the at least one treatment composition is a moisturizing composition sweet almond oil, safflower oil, cetearyl alcohol, algae extract, oat beta glucan, and glycerol.

22. The method of claim 19, further comprising applying at least one of:
a. A cleansing composition;
b. A defense composition;
c. A repair composition; and
d. A sunscreen composition.

23. The method of claim 22, further comprising applying:
a. A cleansing composition;
b. A defense composition;
c. A repair composition;
d. A moisturizing composition;
e. A sunscreen composition; and
f. A cleansing composition.

24. The method of claim 1, wherein the category is assigned a unique identifier.

25. The method of claim 1, wherein the identifier is selected from the group consisting of a color, a symbol, a number, a letter, an alphanumeric code, a sound, a fragrance, a texture, a pattern, and combinations thereof

26-40. (canceled)
